(19) United States
(12) Patent Application Publication
(10) Pub. No.: US 2003/0215470 A1
(43) Pub. Date: Nov. 20, 2003

(54) Compositions and methods for preparing dispersions of thickened oils

(76) Inventors: James M. Wilmott, Shoreham, NY (US); Michael Ross, Dix Hills, NY (US); Duncan T. Aust, Carson City, NV (US); Timothy K. Crawford, Channel Islands, CA (US)

Correspondence Address: Robert C Sullivan, Darby & Darby, Post Office Box 5257, New York, NY, 10150-5257, US

(21) Appl. No.: 10/332,392

(22) PCT Filed: Jul. 9, 2001
(86) PCT No.: PCT/US01/21543

(51) Int. Cl. A61K 31/716; A61K 9/00

(52) U.S. Cl. 424/400; 514/60

(57) ABSTRACT

Disclosed are methods for preparing surfactant free dispersions of hydrophobic fluids and hydrophobic rheological modifying agents which are used to modify the aesthetic properties of cosmetic and pharmaceutical topical compositions. The dispersions are prepared using high pressure/high shear methods. In particular, the dispersions comprise polyisobutene gel or silicone gel.

### FIELD OF THE INVENTION

The present invention relates to methods for preparing surfactant-free oil-in-water dispersions containing a hydrophobic fluid, a hydrophobic rheological modifying agent for the hydrophobic fluid, and an aqueous phase comprising water and water compatible components. The present invention further relates to compositions comprising the surfactant-free oil-in-water dispersions prepared by the method of the invention. The dispersions provide unique aesthetic and water resistant properties to cosmetic and/or dermatological compositions into which the dispersions are incorporated.

### BACKGROUND OF THE INVENTION

Most topical preparations for cosmetic, personal care, over-the-counter or prescription use contain a wide variety of physiologically active ingredients and/or aesthetic modifiers. Physiologically active ingredients cause a physical change to the surface to which they are applied. Examples of such ingredients include alpha hydroxy acids, antioxidants and vitamins. Aesthetic modifiers provide the composition with a defined physical characteristic such as the degree of moisturization, oil content, and physical form of the composition. Some examples of aesthetic modifiers include silicone fluids and derivatives, waxes, botanical (vegetable) oils, hydrocarbon-based oils, esters and fragrances. Some of these ingredients impact the textual, visual, tactile or olfactory properties of the preparation while others are used to protect the preparation from chemical oxidation or microbial contamination.

The physiologically active ingredients and/or aesthetic modifiers possess varying degrees of polarity, and have a spectrum of solubilities ranging from complete oil solubility to complete water solubility; thus these ingredients may not be completely soluble or compatible with all vehicles into which they are incorporated, e.g., oil-based hydrophobic ingredients and a water-based vehicle. As a result, products containing these physically incompatible mixtures may exhibit poor delivery of the active ingredients, have poor tactile properties, and/or may be thermodynamically unstable and have a commercially unacceptable shortened shelf life. Thus, alternative methods to simply mixing these ingredients in water are required.

Non-water based solvents, such as silicone or hydrocarbon based materials of varying polarity, can be used as vehicles for hydrophobic physiologically active materials or aesthetic modifiers. However, these preparations are typically not cosmetically elegant and the non-water based solvents can cause unwanted side effects such as irritation or damage to the epithelial surfaces to which they are applied.

To overcome the drawbacks associated with simple mixtures of aqueous or non-water based solvents, stable emulsions of the physiologically active ingredient and aesthetic modifier may be prepared. These emulsions form either spherical micelles of hydrophobic liquid materials in water or spherical droplets of water in a hydrophobic fluid. Emulsions are typically prepared by first creating the oil phase and water phases, mixing the two phases together, and combining them with one or more emulsifying agents which are incorporated into either or both the water and oil phases. Emulsifiers are surface active agents (surfactants) which reduce the surface tension between the oil and water phases thereby making the combination of the two phases more stable.

Standard emulsion preparations are costly to manufacture due to a variety of factors including the high amount of energy required to heat the preparation, the specialized equipment required to process the emulsion such as specialized pumps and cooling/heating equipment and longer processing times. Emulsions are generally prepared by heating the oil and water phases to elevated temperatures exceeding 70-75° C. before combining, then slowly cooling the combined phases to ensure the development of the crystalline and liquid crystalline structures which give the emulsion its characteristic properties. Heat sensitive actives, fragrances, preservatives and the like, are added at a temperature of less than 40° C. and the composition is cooled to room temperature. The emulsions usually have a homogeneous, opaque, white appearance, and a smooth or pleasant feel when applied to the skin or other epithelial surface.

However, there are a number of limitations in formulating surfactant-based emulsions. For example, each time the oil or water phase is changed, the emulsifiers need to be rebalanced in order to provide the correct hydrophilic/lipophilic balance (HLB). The incorporation of additional materials using conventional techniques can also adversely affect surface tension, leading to instability of the final product. The levels of surfactant present in emulsified preparations also presents problems. Surfactants strip protective layers from the lipid barrier of the skin or the lipid bilayer of epithelial cell membranes leaving the skin tissue vulnerable to further injury. Thus, the surfactants themselves can cause irritation or the damaged barrier will permit the passage of other materials that can cause irritation or increase skin sensitivity and allergic reactions (see, e.g., Effendy I, Maibach H I, Contact Dermatitis 1995 October; 33(4);217-25; Barany E, Lindberg M, Loden M, Contact Dermatitis 1999 February;40(2):98-103).

Unfortunately, many hydrophobic active ingredients and aesthetic modifiers used in conventional topical formulations are not easily processed into emulsions. These agents are readily destabilized in emulsions, due either to surfactant levels or the processing conditions. One example is hydrogenated polyisobutene, a highly desirable and elegant cosmetic ingredient with excellent feel and wear properties which has broad application in a variety of skin care and make-up products (see, e.g., U.S. Pat. Nos. 5,266,321 and 6,013,247). Typically, hydrogenated polyisobutene (also known as liquid isoparaffin) is emulsified into creams and lotions using surfactants. However, surfactants reduce the longer lasting wear characteristics of this material.

Prolonged heating of the water and oil phases can thermodynamically modify the active ingredients or can kinetically accelerate the reaction of the active with other agents in the emulsion or with air if the material is oxygen sensitive. Moreover, lowering the surface tension of topical compositions generally increases the surface exposure of the active or sensitive aesthetic modifiers to oxygen and other destabilizing materials. For example, the instability of unsaturated fatty acids, used as aesthetic modifiers, leads to color changes and malodors in the compositions containing these ingredients. Since the time between manufacturing and sale of a cosmetic product is typically several weeks, products are no longer “fresh” or effective since the active ingredients have degenerated or deteriorated. To offset instability problems, many other materials such as chelating agents, antioxidants and masking agents are usually included in the formulation.

Emulsions may be prepared using high shear conditions to obtain a particle size small enough for maximum stability. Current equipment and procedures are only able to reduce the average particle size to about 3-5 microns in surfactant-based emulsions. However, high shear processing can introduce unwanted air into the formulation, creating changes in density and leading to problems with reproducibility of the manufacturing process and instability of the product. Often the difference in a single parameter such as heating, cooling or mixing rates, is significant enough to cause the product to be outside the established optimum specifications. These batches then have to be either discarded or reworked. The lack of reproducibility can also affect product performance and end user satisfaction. Because products from different batches may have different aesthetic properties, these will be perceived by the end user as a lack of quality and will ultimately lead to consumer dissatisfaction or reduced compliance.

Certain materials are added to cosmetic compositions to enhance the qualities of aesthetic modifying agents. For example, it is often desirable to thicken or increase the viscosity of the products for enhanced tactile sensation or improved wear characteristics (smoother feel, longer wear). These rheological modifying agents, also known as gelling or thickening agents, are added to modify the hydrophobic components of the compositions. The viscosity of various hydrophobic, hydrocarbon materials can be modified, for example, by the addition of di- and tribolock copolymers based on polyalkylene styrene polymers (see, e.g., U.S. Pat. No. 5,221,534). The viscosity of various hydrophobic, silicone materials can be modified through the addition of a crosslinked siloxane elastomer as described in U.S. Pat. No. 5,760,116.

However, these thickened materials present problems in certain processing methods, such as high shear processing. For example, low molecular weight silicone fluids which are thickened with a silicone elastomer to create a gel structure, and which are subjected to high shear conditions, typically increase in viscosity as the amount of gel is increased (see, e.g., U.S. Pat No. 5,998,542). Unfortunately, the high viscosity of this highly desirable class of molecules renders them difficult to use in compounding finished goods.

At present, there is no simple, effective and reproducible method available for the preparation of surfactant free, stable, low viscosity oil-in-water emulsions for the preparation of cosmetic compositions.

It is therefore an object of the invention to provide a method of preparing stable, surfactant-free, low viscosity oil-in-water dispersions comprising hydrophobic physiologically active materials or hydrophobic aesthetic modifiers which are easily formulated into topical cosmetic and pharmaceutical compositions.

### SUMMARY OF THE INVENTION

The present invention provides a method for preparing a high pressure/high shear oil-in-water dispersion comprising a hydrophobic fluid, a hydrophobic rheological modifying agent and aqueous phase. The aqueous phase may comprise in addition to water, water compatible additives and or modifiers. The method comprises the steps of first mixing the hydrophobic fluid and the hydrophobic rheological modifying agent to form a hydrophobic phase, and then mixing the hydrophobic phase with the aqueous phase, and subjecting the hydrophobic/aqueous mixture to high pressure, high shear or high pressure/high shear mixing conditions to form a stable oil-in-water dispersion having a particle size of from about 50 to 1000 mn.

According to the method of the invention, the dispersions of the present invention are produced by mixing from about 1 to 70 wt % (based on total weight of the dispersion), and preferably from about 20 to 50 wt % of a hydrophobic physiologically active or aesthetic modifying fluid agent or mixtures thereof, which has been thickened or formed into a gel by the addition of a hydrophobic rheological modifying agent, with from about 50 to 99 wt % of an aqueous phase (all based on total wt of the mixture) and processing the mixture under high pressure, high shear, or high pressure/high shear conditions until a dispersion having a particle size of from about 50 to 1000 nm, preferably from about 250 to 500 nm, is obtained.

The method of the invention produces a surfactant-free dispersion having a low viscosity, in the range of about 10,000 cps or less, preferably about 5000 cps or less, and most preferably about 1000 cps or less.

In one embodiment of the method of the invention, the hydrophobic fluid is a hydrogenated polyisobutene and the hydrophobic rheological modifying agent is a polyalkylated styrene copolymer. The dispersion comprises about from 0.1 to 70 wt %, and preferably about 5 to 50 wt % of hydrogenated polyisobutene and about 1 to 40 wt % and preferably about 1-15wt % of a blend of polyalkylated styrene copolymers.

Accordingly, the present invention provides a method for preparing a high pressure/high shear dispersion comprising hydrogenated polyisobutene fluid and a polyalkylated styrene copolymer in an aqueous phase, wherein the method comprises the steps of mixing hydrogenated polyisobutene and polyalkylated styrene copolymer to form the hydrophobic phase, and then adding the aqueous phase to the hydrophobic phase and subjecting the hydrophobic/aqueous mixture to high pressure, high shear or concurrent high pressure/high shear mixing to form a stable dispersion that has a particle size in the range of from about 50 to 1000 nm and a specific gravity of 0.8 to 1.00.

In another embodiment of the method of the invention, the hydrophobic agent is a volatile silicone fluid and the hydrophobic rheological modifying agent is a crosslinked siloxane elastomer. The volatile silicone fluid is present in an amount from 1 to 50 wt %; and preferably from about 10 to 40 wt % of the total dispersion. The siloxane elastomer is present in an amount of from about 0.1 to 20 wt %; preferably from about 0.5 to 10 wt % of the total dispersion. The ratio of silicone fluid to silicon elastomer is from about 1:1 to 35:1, preferably from about 6:1 to 32:1. The two components are present in an amount of from about 1 to 50 % of the total dispersion, and preferably from about 10 to 40%.

According to this embodiment of invention, the method comprises the steps of mixing a mixture of a volatile silicone fluid and a siloxane elastomer with an aqueous phase, and subjecting the hydrophobic/aqueous mixture to high pressure/high shear mixing to form a stable dispersion with a particle size of from about 50 to 1000 nm and a specific gravity of 0.8 to 1.00.

The present invention also provides a composition comprising a dispersion prepared by the method of the invention; and a base composition comprising water and a hydrophilic rheological modifying agent. The mixture of water and hydrophilic rheological modifying agent forms a gelled water matrix. According to this embodiment of the invention, the composition comprises the dispersion of a mixture of a hydrophobic phase comprising a mixture of a hydrophobic fluid and a hydrophobic rheological modifying agent, and an aqueous phase, which has been subjected to high pressure, high shear, or high pressure/high shear conditions until a dispersion having a particle size of from about 50 to 1000 nm, preferably from about 250 to 500 nm, is obtained. The dispersion is present in an amount of from about 1 to about 90 wt % and preferably from about 1 to about 40 wt % of the composition. The base composition is present in an amount of from about 10 to 99 wt % of the composition.

The base composition typically comprises from about 0.001 to about 50% and preferably from about 0.01 to about 10%, and more preferably from about 0.1 to about 5% by weight of hydrophilic rheological modifying agents. The base composition typically comprises from about 0.001 to about 99.99%, preferably from about 1 to about 99.99%, and more preferably from about 20 to about 99.99% by weight of water.

The composition may further include other surfactant-free oil-in-water dispersions or other additives/modifiers such as fragrance, chelating agents, colorants and antioxidants as required for the final product.

The composition may be prepared by mixing the dispersion with the aqueous base composition using methods known in the art.

In one aspect of this embodiment, the composition comprises a dispersion of gelled polyisobutene, prepared from hydrogenated polyisobutene fluid and a polyalkylated styrene copolymer. In another aspect of this embodiment, the composition comprises a dispersion of silicone gel prepared from a volatile silicone fluid a crosslinked siloxane elastomer.

Suitable hydrophilic rheological modifying agents include hydrophilic gelling agents including carboxyvinyl polymers, acrylic copolymers, polyacrylamides, polysaccharides, natural gums and clays, or phosphorylated starch derivatives.

The hydrophilic rheological modifying agent is present in the composition in an amount of from about 0.001 to about 50% and preferably from about 0.01 to about 10%, and more preferably from about 0.1 to about 5 wt %.

The base composition typically comprises from about 0.001 to about 99.99%, preferably from about 1 to about 99.99%, and more preferably from about 20 to about 99.99% by weight of water.

The composition may further comprise other additives such as physiological actives and/or aesthetic modifiers as required or suitable for the preparation of topical compositions for the treatment of dermal, anal, oral, vaginal, nasal and opthalmic disorders. Such additives are typically mixed with the dispersion and other ingredients using processes and equipment known in the art.

### BRIEF DESCRIPTION OF THE DRAWING

FIG. 1 is a comparative analysis of the water resistance of sunscreen compositions containing the dispersions of the invention.

### DETAILED DESCRIPTION OF THE INVENTION

It has been surprisingly found that stable oil-in-water dispersions can be prepared when a thickened hydrophobe and water are mixed together under conditions of high pressure and high shear to produce a dispersion that has a particle size of 50 to 1000 nm. Compositions incorporating the dispersions prepared by the method of the invention have a unique tactile sensation when applied topically. The compositions have superior performance and aesthetic properties including long lasting wear properties and enhanced tactile properties, e.g., a lighter, less oily feel, compared to materials prepared by prior art methods which typically contain surfactants as emulsifiers.

For purposes of the invention, “surfactant-free” means that the materials used in the compositions of the inventions do not appreciably reduce the surface tension of the aqueous phase. The dispersions of the present invention may contain agents that help to initiate the micellularization process, for example, phospholipids. These agents may be present in an amount of from about 0.01 to about 5.0 wt %. Suitable agents have low dissociation constants of about in the range of about of 10⁻¹⁰ to 10⁻³⁰ M, and at low oil concentrations do not form micelles, but form very stable lipid bilayers. Phospholipids also appear to increase the surface tension of the composition when drying. The composition prepared is thus substantially free of emulsifying surfactants. The composition preferably comprises less than about 3% by weight and more preferably less than about 1% by weight of emulsifying surfactants, based upon 100% weight of total composition.

The term “dispersion” as used herein refers to a suspension of liquid or solid particles of colloidal size or larger in a liquid medium. Encompassed by the term particles are micelles. Generally, the dispersion contains suspended particles, such as oil particles (or oil droplets), having a diameter less than about 1000 nm. The diameter of the suspended particles preferably ranges from about 50 nm to about 1000 nm and more preferably from about 250 to about 500 nm. Preferably, the suspended particles contain one or more lipophilic materials. The suspended particles may have a charge as determined by zeta potential measurements.

The dispersion containing the suspended particles generally contains from about 0.01 to about 70% by weight of suspended particles, based upon 100% weight of total dispersion. Preferably, the dispersion contains from about 1.0 to about 60% by weight of suspended particles, based upon 100% weight of total dispersion.

A preferred method of processing the oil or hydrophobic phase with water is through the use of high pressure, high shear mixing or high pressure/high shear mixing. The high pressure, high shear mixing or high pressure/high shear mixing may be performed using suitable equipment including homogenizers, microfluidizers and ultrasonic mixers, and mills, such as a Microfluidizer, DeBee high pressure homogenizer, a french press and a Gaulin homogenizer or “Rotor Stator” devices such as a Symex mill, a Silverson mill and a Ross mill. The preferred pressure for preparing the dispersion is between about 11,000 psi to about 25,000 psi with a desired shear that creates an average particle size of between about 50 nm to about 1000 nm.

Typically, the dispersions of the current invention are prepared at ambient conditions. However, the temperature of the hydrophobic fluid gel can be increased to as high as 70° C. to reduce the viscosity of the gel, thereby facilitating the break up of the hydrophobic phase and creating small particles more readily.

The particle size is controlled by the number of passes the dispersion is subjected to during the high pressure/high shear process. The dispersion is mixed until the desired particle size of <1000 mn is achieved.

The method of the invention permits the formation of stable dispersions with or without the presence of traditional emulsifiers such as surfactants. Surfactant-free compositions are preferred because they reduce or eliminate the problems created by the presence of a surfactant. Furthermore, the system requires no heating and thereby maintains the integrity of sensitive physiologically active materials and aesthetic modifiers. High pressure/high shear dispersions find applications in cosmetic, personal care, over-the-counter (OTC), Rx, nutritional and food products.

The surfactant-free dispersions prepared by the method of the present invention demonstrate improved stability and wear characteristics. The very small particle size of the dispersions produce a more uniform film on surfaces to which they are applied (improved film deposition) than prior art compositions containing large particle size emulsions.

Because the viscosity of the finished good products may be conveniently preset by the rheological modifying agent and the dispersions of the invention are stable and maintain the desired particle size, the compositions of the invention may be prepared by methods that reduce manufacturing costs by reducing processing time and energy costs resulting in lower capital investment in equipment. These methods permit easier scale up to manufacturing, and result in much more consistent reproducibility than prior art methods, causing less wasted batches and work-off. The ingredients may be simply mixed using methods that require little energy. The method of the invention also provides greater flexibility in obtaining formulations, substituting ingredients, and allows the formulator to disregard hydrophilic-lipophilic balance (HLB) rebalancing which is often a problem when changes are made to oil-in-water formulations.

A hydrophobic active ingredient or hydrophobic aesthetic modifying agent of the present invention is one which has a non polar property which makes it essentially insoluble in water or water and polar solvent solutions. Hydrophobic active ingredients and hydrophobic aesthetic modifying agents of the present invention include, but are not limited to, partially and fully hydrophobic active ingredients and partially and fully hydrophobic aesthetic modifying agents. For example, hydrophobic active ingredients encompassed by the present invention include anti-acne agents, anti-inflammatory agents, analgesics, antiedemal agents, antipsoriatic agents, antifungal agents, skin protectants, sunscreen agents, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, antibiotics, anesthetics, steroids, tissue healing substances, tissue regenerating substances, ceramides and any combination of any of the foregoing. Preferred anti-acne agents include, but are not limited to, retinoic acid, azelaic acid.

Suitable anti-inflammatory agents include, but are not limited to bisabolol.

Suitable analgesics include, but are not limited to menthylsalicylate, turpentine oil, capsicum, methyl nicotinate, and any combination of any of the foregoing.

Suitable antiedmal agents include, but are not limited to, caleipotriene, coal tar, anthralin, vitamin A, and any combination of any of the foregoing.

Suitable antifungal agents include, but are not limited to cocoa butter, cod liver oil, dimethicone, lanolin (in combination), mineral oil, peruvian balsam, petrolatum, shark liver oil, Vitamin A, Vitamin E, White petrolatum.

Suitable sunscreen agents include, but are not limited to, ethyl hexyl methoxycinnamate, ethyl hexyl para amino benzoic acid ester, homosalate, octoacrylene and any combination of any of the foregoing.

Suitable antioxidants include, but are not limited to, scavengers for lipid free radicals and peroxyl radicals, quenching agents, and any combination of any of the foregoing. Suitable antioxidants include, but are not limited to, tocopherol, beta carotene, vitamin A, ubiquinol, azelaic acid, ubiquinone and any combination of any of the foregoing.

Suitable vitamins include, but are not limited to, vitamin E, vitamin A, vitamin A palmitate, vitamin D, vitamin F, vitamin E acetate, derivatives of any of the foregoing, and any combination of any of the foregoing.

The composition of the current invention includes at least one or more hydrophobic aesthetic modifying agents. An aesthetic modifying agent is a material which imparts desirable tactile, olfactory, taste or visual properties to the surface to which it is applied. The aesthetic modified generally is a hydrophobe, preferably the hydrophobe is a fluid.

The hydrophobic component may be derived from animals, plants, or petroleum and may be natural or synthetic. Preferred hydrophobic components are substantially water-insoluble, more preferably essentially water-insoluble. Preferred hydrophobic components are suitable for conditioning the skin. Suitable oil components include, but are not limited to, natural oils, such as coconut oil; hydrocarbons, such as mineral oil and hydrogenated polyisobutene; fatty alcohols, such as octyldodecanol; esters, such as C₁₂₋₁₅ alkyl benzoate; diesters, such as propylene glycol dipelargonate; triesters, such as glyceryl trioctanoate; sterol derivatives, such as lanolin; animal waxes, such as beeswax; plant waxes, such as carnauba; mineral waxes, such as ozokerite; petroleum waxes, such as paraffm wax; synthetic waxes, such as polyethylene; and mixtures thereof. Suitable oil components may also be silicones including, but not limited to, volatile silicones such as cyclomethicone; polymeric silicones such as dimethicone; alkylated derivatives of polymeric silicones, such as cetyl dimethicone and lauryl trimethicone; hydroxylated derivatives ofpolymeric silicones, such as dimethiconol; and mixtures thereof. Examples may also include organopolysiloxanes such as polyalkylsiloxanes, alkyl substituted dimethicones, cyclomethicones, trimethylsiloxysilicates, dimethiconols, polyalkylaryl siloxanes, and mixtures thereof. More preferred for use herein are polyalkylsiloxanes and cyclomethicones. Preferred among the polyalkylsiloxanes are dimethicones.

A preferred example of such a silicone fluid is volatile silicone fluid. A suitable gelled volatile silicone fluid is sold by under the trade name SFE 839^{ä} (General Electric Company). Other examples of suitable elastomers and hydrophobic fluids are described in U.S. Pat. Nos. 5,998,542 and 5,760,116.

Preferably the non-aqueous hydrophobic aethetic modifying agent is a hydrogenated polyisobutene, available as Polysynlane^{â} (Collaborative Laboratories, Inc., East Setauket, N.Y.) manufactured by NOF Corporation.

The dispersion of the current invention includes at least one or more hydrophobic rheological modifying agents (also known as hydrophobic thickening or gelling agents). A hydrophobic thickening agent is a material that changes the rheological properties of the hydrophobic fluid typically by increasing the viscosity of the fluid. The hydrophobic thickening or gelling agents are typically polymers or copolymers. They include, but are not limited to polyalkylene styrene di- and triblock copolymers, silicone elastomer, alkylated polyethylenes and alkylated polysaccharide polymers or copolymers.

Preferred polyalkylene styrene copolymers include, but are not limited to di- and triblock copolymers including, hydrogenated butylene/etliylene/styrene copolymer and hydrogenated ethylene/propylene/styrene copolymer.

Preferred silicone elastomers include, but are not limited to, dimethicone/vinyl dimethicone crosspolymer.

Preferred alkylated polyethylenes include, but are not limited to, C₃₀₋₄₀ polyethylenes, sold under the trade name Performacids (New Phase, Piscataway, N.J.).

Preferred allylated polysaccharide copolymers include, but are not limited to C₁₋₅ alkyl galactomannan. Galactomannan is a natural non-ionic polysaccharide onto which short chain alkyl groups are grafted. Preferably the alkyl galactomannan thickening agent is sold under the trade name N-Hance AG (Hercules, Wilmington, Del.).

The poly-alkylated styrene copolymer used in the composition of the invention is typically available in various hydrophobic fluids as GEAHLANE (PENRECO, USA). The copolymer is also available in hydrogenated polyisobutene as Polysynlane^{â} Gel (Collaborative Laboratories, Inc. East Setauket, N.Y.).

The composition of the current invention includes at least one or more hydrophilic rheological modifying agents (also known as hydrophilic thickening or gelling agents), including phosphorylated starch derivatives and co-thickening agents.

The term “phosphorylated starch derivative” includes, but is not limited to, starches containing a phosphate group. Suitable phosphorylated starch derivatives include, but are not limited to, hydroxyalkyl starch phosphates, hydroxyalkyl distarch phosphates, and any combination of any of the foregoing. Non-limiting examples of hydroxyalkyl starch phosphates and hydroxyalkyl distarch phosphates include hydroxyethyl starch phosphate, hydroxypropyl starchphosphate, hydroxypropyl distarch phosphate, and any combination of any of the foregoing. According to a preferred embodiment, the base composition comprises a hydroxyalkyl distarch phosphate and more preferably hydroxypropyl distarch phosphate.

Suitable co-thickening agents include, but are not limited to, carbohydrate based thickening agents, polymeric and copolymeric thickening agents, inorganic thickening agents, protein thickening agents, polypeptide thickening agents, and any combination of any of the foregoing.

Non-limiting examples of suitable carbohydrate based thickening agents include algin and derivatives and salts thereof, such as algin, calcium alginate, propylene glycol alginate, and ammonium alginate; carrageenan (Chondrus crispus) and derivatives and salts thereof, such as calcium carrageenan and sodium carrageenan; agar; cellulose and derivatives thereof, such as carboxymethyl hydroxyethylcellulose, cellulose gum, cetyl hydroxyethylcellulose, hydroxyethylcellulose, hydroxypropylcellulose, hydroxypropyl methylcellulose, methylcellulose, ethylcellulose, and cellulose gum; chitosan and derivatives and salts thereof, such as hydroxypropyl chitosan, carboxymethyl chitosan, and chitin; gellan gum; guar (Cyanopsis tetragonoloba) and derivatives thereof, such as guar hydroxypropyltrimonium chloride and hydroxypropyl guar; hyaluronic acid and derivatives thereof; dextran and derivatives thereof; dextrin; locust bean (Ceratonia siliqua) gum; mannans and derivatives thereof, such as C₁₋₅ alkyl galactomannan; starches, such as starch polyacrylonitrile copolymer-potassium salt and starch polyacrylonitrile copolymer-sodium salt; pectin; sclerotium gum; tragacanth (Astragalus gummifer) gum; xanthan gum and derivatives thereof; and any combination of any of the foregoing.

Non-limiting examples of suitable polymeric and copolymeric thickening agents include acrylates, methacrylates, polyethylene and derivatives thereof, and any combination of any of the foregoing. Suitable acrylates and methacrylates include, but are not limited to, carbomer and derivatives and salts thereof, acrylate/C₁₀-C₃₀ alkyl acrylate crosspolymer, acrylate/ceteth-20 itaconate copolymer, acrylate/ceteth-20 methacrylate copolymers, acrylate/steareth-20 methacrylate copolymers, acrylate/steareth-20 itaconate copolymers, acrylate/steareth-50 acrylate copolymers, acrylate/VA crosspolymers, acrylate/vinyl isodecanoate crosspolymers, acrylic acid/acrylonitrogen copolymers, ammonium acrylate/acrylonitrogen copolyrners, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylate/acrylamide copolymer, acrylate copolymers, acrylate/hydroxyester acrylate copolymers, acrylate/octylarylamide copolymers, acrylate/PVP copolymers, AMP/acrylate copolymers, butylester of PVM/MA copolymer, carboxylate vinylacetate terpolymers, diglycol/CHDM/isophthalates/SIP copolymer, ethyl ester of PVM/MA copolymer, isopropyl ester of PVM/MA copolymer, octylacrylamide/acrylate/butyl-aminoethyl methacrylate copolymers, polymethacrylamidopropyltrimonium chloride, propylene glycol oligosuccinate, polyvinylcaprolactam, PVP, PVP/dimethylamino-ethylmethacrylate copolymer, PVP/DMAPA acrylate copolymers, PVP/carbamyl polyglycol ester, PVP/VA copolymer, PVP/VA vinyl propionate copolymer, PVP/vinylcaprolactam/DMAPA acrylate copolymers, sodium polyacrylate, VA/butyl maleate/isobornyl acrylate copolymers, VA/crotonates copolymer, VA/crotonates vinyl neodecanoate copolymer, VA crotonates/vinyl propionate copolymer, vinyl caprolactam/PVP/dimethylamineoethyl-methacrylate copolymer, and any combination of any of the foregoing. The following acronyms relate to: AMP=aminomethyl propanol; DMAPA=dimethylaminopropyl-amine; VA=vinyl acrylates; PVM/MA=copolymer of methyl vinyl ether and maleic anhydride; CHDM=1,4-cycolhexanedimethanol; SIP=sulfoisophthalic acid; PVP=polymer of 1-vinyl-2 pyrrolidine monomers.

Non-limiting examples of suitable inorganic thickening agents include clays and derivatives thereof, silicates, silicas and derivatives thereof, and any combination of any of the foregoing. Suitable clays and derivatives thereof include, but are not limited to, bentonite and derivatives thereof, such as quaternium-18 bentonite; hectorite and derivatives thereof, such as quaternium-18 dectorite; montmorillonite; and any combination of any of the foregoing. Suitable silicates include, but are not limited to, magnesium aluminum silicate, sodium magnesium silicate, lithium magnesium silicate, tromethamine magnesium aluminum silicate, and any combination of any of the foregoing. Suitable silicas and derivatives thereof include, but are not limited to, hydrated silica, hydrophobic silica, and any combination of any of the foregoing.

Suitable protein and polypeptide thickening agents include, but are not limited to, proteins and derivatives and salts thereof, polypeptides and derivatives and salts thereof, and any combination of any of the foregoing. Non-limiting examples of protein and polypeptide thickening agents include albumin, gelatin, keratic and derivatives thereof, fish protein and derivatives thereof, milk protein and derivatives thereof, wheat protein and derivatives thereof, soy protein and derivatives thereof, elastin and derivatives thereof, silk protein and derivatives thereof, and any combination of any of the foregoing.

Preferred thickening agents include, but are not limited to, carbomer, acrylate/allyl acrylate crosspolymers, acrylate/vinyl isododecanoate crosspolymer, xantham gum, locust bean gum, guar gum, and any combination of any of the foregoing. A more preferred combination of thickening agents comprises carbomer and an acrylate/alkyl acrylate copolymer, such as an acrylate/C₁₀-C₃₀ alkyl acrylate copolymer. According to the International Cosmetic Ingredient Dictionary and Handbook (7^{th} Ed., The Cosmetic, Toiletry, and Fragrance Association), carbomer is a homopolymer of acrylic acid crosslinked with an allyl ether of pentaerythritol, an allyl ether of sucrose, or an allyl ether ofpropylene. The term “acrylate/alkyl acrylate crosspolymer” includes, but is not limited to, copolymers of alkyl acrylates with one or more monomers of acrylic acid, methacrylic acid, or one of their short chain (i.e. C.₁₋₄ alcohol) es ters, wherein the crosslinking agent is, for example, an allyl ether of sucrose or pentaerytritol. Preferably, the alkyl acrylates are C₁₀-C₃₀ alkyl acrylates. Examples of such copolymers include, but are not limited to, those commercially available as Carbopol™ 1342, Carbopol™ 1382, Pemulen™ TR-1, and Pemulen™ TR-2, from Goodrich Specialty Chemicals of Cleveland, Ohio.

The base composition typically comprises from about 0.001 to about 50% and preferably from about 0.01 to about 10%, and more preferably from about 0.1 to about 5% by weight of hydrophilic rheological modifying agents. The base composition typically comprises from about 0.001 to about 99.99%, preferably from about 1 to about 99.99%, and more preferably from about 20 to about 99.99% by weight of water.

The hydropholic rheological modifying agent can be simply mixed with the water using a propellor. However, the process can be accelerated using a colloid mill or homogenizer which rapidly hydrates the thickener.

Phospholipids which may comprise from 0.01% to 8% by weight (preferably 0.01 to 5% by weight) of the dispersion may include Phospholipon 80, 80H (American Lecithin Co., Oxford, Conn.), Basis LP2OH (Ikeda Corp., Japan), and Catemol, a synthetic lipid-like compound (Phoenix Chemicals Inc., Somerville, N.J.).

The topical preparation of the present invention may also include non-hydrophobic active ingredients and non-hydrophobic aesthetic modifiers.

The dispersions of thickened hydrophobic fluids in water described in the present invention can be added to a base containing a suitable hydrated aqueous gelling or thickening agent or blends thereof to impart either the functional benefit or aesthetic property to a finalized finished good. Furthermore, the dispersions of the current invention can be mixed with other stabilized high pressure/high shear dispersions in a base containing a hydrated gelling agent. The combinations of selected dispersions in a base containing a hydrated gelling agent with other water soluble physiologically active or aesthetic modifying agents creates the desired functionality and aesthetic properties to a product intended for use by the consumer.

### EXAMPLES

### Example 1

Process to Produce High Pressure/High Shear Dispersion of Gelled Hydrogenated Polyisobutene, Volatile Silicone and Mineral Oil

Dispersions 1-3 were prepared using the ingredients in Table 1 as follows:

1. The ingredients of Phase A and B were mixed with a propeller blade using moderate speed until homogeneous at room temperature.

2. With continuous mixing, the ingredients of Phase C were combined with the A and B mixture. 3. The mixture was then mechanically sheared with a homogenizer until phase ABC was uniform.

4. Phase ABC was sheared using high pressure/high shear mixing employing pressures of 11,000-25,000 psi until the desired particle size of <1000 nm was reached.

TABLE 1

| Phase | Ingredient | Dispersion 1 | Dispersion 2 | Dispersion 3 |
|---|---|---|---|---|
| A | Deionized water | Qs to 100% w/w | Qs to 100% w/w | Qs to 100% w/w |
| | Germazide^{â} MPB² | 1.25 | 1.20 | 1.60 |
| | Peg 8³ | 5.00 | 5.00 | 5.00 |
| B | Basis LP-20H⁷ | 2.00 | 2.50 | 2.00 |
| | Phospholipon 80H⁸ | 0.75 | 0.50 | 0.25 |
| C | Polysynlane^{â} Lite¹ | 10 | — | — |
| | Polysynlane^{â} Gel² | 30 | — | — |
| | SFE-839_{ä}⁴ | — | 30.00 | — |
| | Versagelä M750⁵ | — | — | 30.00 |
| | DC345 fluid⁶ | — | 4.00 | 5.00 |

1: NOF Corporation, Tokyo, Japan.
2: Collaborative Laboratories, Ltd., Stony Brook, NY.
3: Union Carbide Corp. Houston, TX.
4: General Electric Company.
5: Penreco Chemical Company, Karns City, PA.
6: Dow Corning Company, Midland, MI.
7: The Nisshin Oil Mills, Ltd., Tokyo, Japan.
8: Natterman Phospholipid GMBH, Cologne, Germany.

### Example 2

Preparation of Creams Containing Dispersions

Creams 1-3 were prepared using the ingredients shown in Table 2 as follows:

1. With moderate speed propeller agitation the water in phase A was added to the moisturizing base and mixed until the gel was smooth and homogeneous.

2. The remaining ingredients in phase A were added sequentially and mixed until completely uniform.

3. With slow to moderate paddle blade agitation the ingredients in phase B were sequentially added to phase A and mixed until homogeneous.

4. Sequentially the ingredients in phase C were added to the batch and mixed until completely uniform.

TABLE 2

| Phase | Ingredient | Cream 1 | Cream 2 | Cream 3 |
|---|---|---|---|---|
| A | Moisturizing Base | 66.50 | 41.00 | 35.00 |
| | Water | — | 6.50 | 11.50 |
| | Advanced Moisture | 3.50 | 5.00 | 5.00 |
| | Complexä² | | | |
| | AM 900² | 5.00 | 5.00 | 4.00 |
| B | AM 200² | 7.00 | 12.00 | 12.00 |
| | AM 500² | 3.00 | 6.00 | 6.00 |
| | Dispersion 2 | — | — | 8.00 |
| | Dispersion 1 | 7.00 | 10.00 | 3.00 |
| C | Acullynä44¹ | 1.00 | 1.00 | 0.50 |
| | Butylene Glycol | 1.00 | 3.50 | 3.00 |
| | (BG)⁵ | | | |
| | 50% EA 209 in BG³ | — | — | 2.00 |
| | SanSurf^{â} DMG² | — | 5.00 | — |
| | Seamolient^{â2} | 2.00 | 2.50 | — |
| | HA-Solä 1%² | 2.00 | 2.50 | — |
| | Retinol | — | — | 10.00 |
| | Catezomesä² | | | |
| | Nanocell^{â} EFA² | 2.00 | — | — |
| | Total | 100.00 | 100.00 | 100.00 |
| | Percentage (%) | | | |

1: ISP Corporation, New Jersey. USA.
2: Collaborative Laboratories, Inc., East Setauket, NY.
3: Kobo, Inc., New Jersey, USA.
4: Chisso Corporation. Tokyo, Japan.
5: Kramer, New Jeraey, USA.

### Example 3

Preparation of SPF Creams Containing Dispersions

SPF creams 1 and 2 were prepared using the ingredients in Table 3 as follows:

1. With moderate propeller agitation the water in phase A was slowly added to the moisturizing base and mixed until a smooth homogeneous fluid was obtained.

2. The remaining ingredients in phase A were sequentially added and the product was mixed until completely uniform.

3. The ingredients in phase B were sequentially added to phase A using a paddle blade with slow to moderate agitation. The product was mixed until completely uniform.

TABLE 3

| Phase | Ingredient | SPF Cream 1 | SPF Cream 2 |
|---|---|---|---|
| A | Moisturizing Base | 35.25 | 10.50 |
| | Water | 17.25 | 46.75 |
| | Advanced Moisture Complexä² | 1.00 | 4.00 |
| B | AM 200² | 14.50 | 5.00 |
| | AM 300² | 6.50 | 5.50 |
| | AM 400² | 4.50 | 4.00 |
| | AM 500² | — | 3.00 |
| | Dispersion 1 | 1.00 | 1.00 |
| | Germaben^{â}2¹ | — | 0.25 |
| | Solarease^{â}II² | 20.00 | 20.00 |
| Total | | 100.00 | 100.00 |
| Percentage | | | |

1: ISP Corporation, New Jersey, USA.
2: Collaborative Laboratories, Inc., East Setauket, NY.

### Example 4

Preparation of SPF Moisturizing Creams Containing Dispersions

SPF Moisturizing Creams 1-3 were prepared with the ingredients in Table 4 as follows:

1. With moderate propeller agitation the water in phase A was added to the moisturizing base and mixed until the product was smooth and homogeneous.

2. The remaining ingredients in phase A were sequentially added and mixed until completely uniform.

3. The ingredients in phase B were added to phase A and mixed with a paddle blade agitation at slow to moderate speed until homogeneous.

4. Sequentially the ingredients in phase C were added and mixed with paddle blade agitation until the product was completely uniform.

TABLE 4

| | | SPF Moisturizing | SPF Moisturizing | SPF Moisturizing |
|---|---|---|---|---|
| Phase | Ingredient | Cream 1 | Cream 2 | Cream 3 |
| A | Moisturizing Base² | 38.00 | 40.00 | 41.00 |
| | Water | 8.25 | 6.00 | 5.00 |
| | Advanced Moisture | 1.75 | 2.00 | 2.00 |
| | Complexä² | | | |
| | AM 900² | 6.00 | 6.00 | 6.00 |
| B | AM 200² | 6.00 | 6.00 | 6.00 |
| | AM 300² | 6.00 | 3.00 | 3.00 |
| | AM 500² | 2.50 | — | — |
| | Dispersion 1 | 6.50 | 7.00 | 7.00 |
| C | Acullynä44¹ | — | 1.00 | 1.00 |
| | 25% Celluflow in BG³ | — | 3.50 | 3.50 |
| | Solarease^{â}II² | 20.00 | 20.00 | 20.00 |
| | SanSurf^{â} DMG² | 1.00 | 1.00 | 1.00 |
| | SanSurf^{â} Vitamin E² | 1.00 | 1.00 | 1.00 |
| | Seamolient^{â2} | 1.00 | 1.50 | 1.50 |
| | Total Percentage | 100.00 | 100.00 | 100.00 |

1: ISP Corporation, New Jersey, USA.
2: Collaborative Laboratories, Inc., East Setauket, NY.
3: Chisso Corporation. Tokyo, Japan

### Example 5

Water Resistancy of Sunscreens Containing Dispersions

Sunscreens are intended to protect skin from solar radiation. However, upon application to skin, sunscreens are likely to lose their activity due to contact with external water. Therefore the sunscreens that do not lose their activity in the presence of water have high consumer value. Thus, ascertaining the water-proofing property of sunscreens is very important.

### MATERIALS AND METHODS

Sunscreens containing butyl methoxydibenzoylmethane (Parsol 1789) and octyl methoxycinnamate (OMC) for protection against both UVA and UVB damage to the human skim, formulated with different water resistant gels, were tested.

Known amounts of the sunscreen formulations were spread on “Vitro Skin” (Ims, Inc.) and allowed to dry overnight at room temperature. Vitro skin containing films of sunscreens were placed in a vial containing water and held at 28° C. for 80 min in a water bath. Vitro skin was removed from the vial and placed in another vial containing methanol and sonicated to dissolve remaining sunscreen on the skin and diluted with methanol to a known volume in a volumetric flask and assayed quantitatively via HPLC following established protocols (Deflandre, A, and Lang, G., Intl. J. Cosmet. Sci 10:53-62, (1988); Jiang, R. et al., J. Chromatoar. B Biomed. Appl. 682: 137-145, (1996); Vanquerq. V. et al., J. Chromatogr., 832:137-145(1996)) and the following instrumentation: Waters model 600 E; mobile phase: isocratic, consisting of 60:40:0.1 Acetonitrile:water:phosphoric acid; column temperature 50° C.; injection volume 20.0 μl detection at 330 nm.

### RESULTS AND DISCUSSION

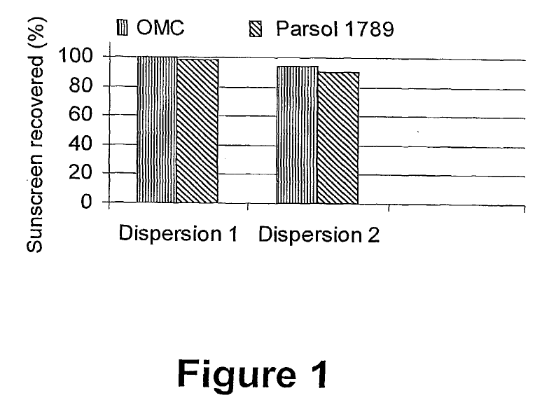

The sunscreen formulations containing Dispersion 1 and Dispersion 2 showed very high water proofing quality. The results are shown in FIG. 1.

### CONCLUSION

The results in FIG. 1 show that sunscreen formulations containing dispersions of the present invention have almost 100% resistance to water as demonstrated by an almost 100% recovery rate of the sunscreen from the applied surface.

All patents, publications, applications, and test methods mentioned herein are hereby incorporated by reference. Many variations of the present invention will suggest themselves to those skilled in the art in light of the above, detailed description. All such obvious variations are within the full intended scope of the appended claims.

What is claimed is:

1. A method for preparing a surfactant-free oil-in-water dispersion comprising a hydrophobic fluid, a hydrophobic rheological modifying agent and an aqueous phase, comprising the steps of mixing the hydrophobic fluid and the hydrophobic rheological modifying agent to form a hydrophobic phase, adding the aqueous phase to the hydrophobic phase, and processing the hydrophobic/aqueous mixture under high pressure/high shear mixing conditions to form a stable oil-in-water dispersion having a particle size of from about 50 to 1000 nm.

2. The method of claim 1, wherein the hydrophobic phase is present in an amount of from about 1% to 70%, and preferably from about 20% to 50% of the total composition, and the aqueous phase is present in an amount of from about 50% to 99% of the total composition.

3. The method of claim 1, wherein the dispersion has a viscosity of about 5000 cps or less, and preferably of 1000 cps or less.

4. The method of claim 1, wherein the hydrophobic fluid is a hydrogenated polyisobutene and the hydrophobic rheological modifying agent is a polyalkylated styrene copolymer.

5. The method of claim 4, wherein the hydrogenated polyisobutene is present in an amount of from about 0.1 to 70 wt %, preferably from about 5 to 50 wt % based on the total weight of the composition.

6. The method of claim 4 wherein the polyalkylated styrene copolymer is present in an amount of from about 0.1 to 40 wt %, preferably from about 1 to 15 wt % based on total weight of the composition.

7. The method of claim 1, wherein the hydrophobic fluid is a volatile silicone fluid and hydrophobic rheological modifying agent is a crosslinked siloxane elastomer.

8. The method of claim 7, wherein the volatile silicone fluid is present in an amount from about 1 to 50 wt %, and preferably from about 10 to 40 wt % of the total composition.

9. The method of claim 7, wherein the siloxane elastomer is present in an amount of from about 0.1 to 20 wt %, and preferably from about 0.5 to 10 wt % of the total composition.

10. The method of claim 7, wherein the ratio of silicone fluid to silicon elastomer is from about 1:1 to 35:1, and preferably from about 6:1 to 32:1.

11. The method of claim 7, wherein the hydrophobic phase is present in an amount of from about 1 to 50% of the total composition.

12. The method of claim 7, wherein the hydrophobic agent is cyclopentasiloxane and the gelling/suspending agent is a dimethicone/vinyl dimethicone crosspolymer.

13. A composition comprising:
a) an oil in water dispersion prepared by the method of claim 1; and
b) a base composition comprising
(i) a hydrophilic rheological modifying agent, and
(ii) an aqueous phase,
wherein the composition has a particle size of less than 1000 microns.

14. The composition of claim 13 wherein the base composition comprises from about 0.01 to about 10% by weight of the hydrophilic rheological modifying agent.

15. The formulation of claim 13 wherein the base composition comprises from about 20 to about 99.99% by weight of water.

16. The composition of claim 13 wherein the dispersion comprises from about 1 to 90% by weight of the total composition.

17. The formulation of claim 13 wherein the base composition comprises from about 10 to about 99% by weight of the total composition.

18. The composition of claim 13, wherein the hydrophilic rheological modifying agent comprises a hydrophilic gelling agent.

19. The composition of claim 18, wherein the hydrophilic gelling agent comprises one or more members selected from the group consisting of carboxyvinyl polymers, acrylic copolymers, polyacrylamides, polysaccharides, natural gums and clays.

20. The composition of claim 13, wherein the rheological modifying agent comprises a phosphorylated starch derivative.

21. The composition of claim 20, wherein the phosphorylated starch derivative is hydroxypropyl distarch phosphate.

22. The composition of claim 13, wherein the rheological modifying agent is selected from the one or more members of the group consisting of sodium hyaluronate, acrylates/C₁₀-C₃₀ alkyl acrylate crosspolymer, xanthum gum, cholesterol, hydroxypropyl distarch phosphate, carbomer, guar hydroxy propyltrimonium chloride, hydroxypropyl guar and sodium hydroxypropyl starch phosphate.

23. A composition comprising an oil in water dispersion prepared by the method of claim 4.

24. A composition comprising an oil in water dispersion prepared by the method of claim 7.

25. The composition of claim 13 for topical, anal, vaginal, ophthalmic, nasal or otic application.
